(19) United States
(12) Patent Application Publication
(10) Pub. No.: US 2017/0049344 A1
(43) Pub. Date: Feb. 23, 2017

(54) PULSE WAVE SENSOR AND PULSE WAVE MEASUREMENT MODULE

(71) Applicant: Rohm Co., Ltd., Kyoto (JP)

(72) Inventor: Koji Terumoto, Kyoto (JP)

(73) Assignee: Rohm Co., Ltd., Kyoto (JP)

(21) Appl. No.: 15/307,459

(22) PCT Filed: Apr. 30, 2015
(86) PCT No.: PCT/JP2015/062993

§ 371 Date: Oct. 28, 2016

(30) Foreign Application Priority Data
May 2, 2014 (JP) 2014-095367
Oct. 16, 2014 (JP) 2014-211684

(51) Int. Cl. A61B 5/024; A61B 5/00

(52) U.S. Cl. CPC A61B 5/02416; A61B 5/6824; A61B 5/7225; A61B 5/742

(57) ABSTRACT

A pulse wave sensor includes a light sensor unit arranged to irradiate a living body with light from a light emitting unit and to detect reflected light or transmitted light from the living body by a light receiving unit, so as to generate a current signal corresponding to received light intensity, a pulse drive unit arranged to turn on and off the light emitting unit at a predetermined frame frequency and duty, a transimpedance amplifier arranged to convert the current signal into a voltage signal, and a mounting determination unit arranged to perform mounting determination by comparing an OFF voltage signal obtained by the transimpedance amplifier during an OFF period of the light emitting unit with a predetermined first threshold voltage.

### TECHNICAL FIELD

The present invention relates to a pulse wave sensor.

### BACKGROUND ART

Conventionally, there is known a pulse wave sensor (so-called photoelectric pulse wave sensor), which irradiates a living body (such as an arm or a finger of a subject) with light from a light emitting unit and detects a pulse wave of the subject based on received light intensity of light after passing through the living body. In this type of the pulse wave sensor, the received light intensity varies due to beat of the subject, and hence various information of the pulse wave (such as a pulse rate of the subject) can be obtained based on characteristics of the pulse wave signal corresponding to the received light intensity (such as a variation period of the pulse wave signal).

Note that, as an example of the background art related to the above description, there is Patent Document 1.

### PRIOR ART DOCUMENTS

### Patent Documents

Patent Document 1: JP-A-05-161615

### DISCLOSURE OF THE INVENTION

### Problem to be Solved by the Invention

Here, in order to correctly obtain a pulse rate or the like of the subject, it is necessary to correctly mount the pulse wave sensor on the arm or the finger of the subject. However, a conventional pulse wave sensor performs measurement without determining whether or not it is mounted on the living body and outputs a result of the measurement as a parameter value.

Therefore, for example, if the pulse wave sensor is powered on before being mounted on a living body, it may output an insignificant parameter value so as to be an unnatural operation state. In addition, for example, if the pulse wave sensor is incorrectly mounted on a living body, it may output an incorrect parameter value so as to be an improper operation state.

Note that as a mounting determination method of the pulse wave sensor, for example, there is a method of detecting an amplitude level of a pulse wave. This method includes allowing a light emitting unit to emit light at a predetermined light emission intensity, reading directly an amplitude of a pulse wave signal (i.e. a difference between a maximum signal value and a minimum signal value), and determining mounted/unmounted on a living body based on whether or not the read amplitude is higher than a predetermined threshold value.

However, supposing that a period of a pulse wave during rest corresponds to 1 Hz, it takes approximately at least one second and usually two to three seconds to directly read the amplitude of the pulse wave signal. In addition, in order to increase accuracy of the mounting determination, it is considered to repeat the reading of the amplitude n times (n≧2). In this case, the time necessary for the mounting determination becomes n times the time described above (i.e. 2n to 3n seconds, which is approximately 10 seconds in ordinary cases).

In addition, for example, when the pulse wave sensor is left without being mounted on a living body, the pulse wave signal is fixed to a reference voltage in a situation where ambient light does not change, while its amplitude varies in a situation where the ambient light varies. In addition, when the pulse wave sensor is moved without being mounted on a living body (e.g. when it is carried by a hand), the amplitude also varies. Accordingly, it is difficult to determine an unmounted state based on the amplitude of the pulse wave signal.

On the other hand, it is considered to dispose an additional mounting sensor (such as a proximity sensor) for detecting mounting of the pulse wave sensor on a living body. However, in this case, adding of the mounting sensor causes more complicated control, increase in the number of components, increase in cost, or increase in size.

In view of the above-mentioned problem found by the inventors, it is an object of the present invention to provide a pulse wave sensor that can quickly and correctly determine mounted/unmounted on a living body.

### Means for Solving the Problem

In order to achieve the above-mentioned object, a pulse wave sensor according to one aspect of the present invention includes:

a light sensor unit arranged to irradiate a living body with light from a light emitting unit and to detect reflected light or transmitted light from the living body by a light receiving unit, so as to generate a current signal corresponding to received light intensity;

a pulse drive unit arranged to turn on and off the light emitting unit at a predetermined frame frequency and duty;

a transimpedance amplifier arranged to convert the current signal into a voltage signal; and

a mounting determination unit arranged to perform mounting determination by comparing an OFF voltage signal obtained by the transimpedance amplifier during an OFF period of the light emitting unit with a predetermined first threshold voltage (a first structure).

In addition, in the first structure described above, the first threshold voltage may be set to a voltage value lower than a reference voltage of the transimpedance amplifier (a second structure).

In addition, in the first or second structure, the mounting determination unit may perform the mounting determination by comparing an ON voltage signal obtained by the transimpedance amplifier during an ON period of the light emitting unit with a predetermined second threshold voltage and a predetermined third threshold voltage lower than the second threshold voltage (a third structure).

In addition, the third structure described above may further include a luminance adjustment control unit arranged to adjust luminance of the light emitting unit by comparing a voltage value based on the ON voltage signal obtained by controlling the pulse drive unit to turn on and off the light emitting unit with a predetermined threshold voltage for adjustment, in which the second threshold voltage may be higher than the threshold voltage for adjustment, while the third threshold voltage may be lower than the threshold voltage for adjustment (a fourth structure).

In addition, in the third or fourth structure described above, the mounting determination unit may change a first count number or a second count number according to whether or not both the OFF voltage signal and the ON voltage signal satisfy a mounting determination condition, so as to perform determination of mounted/unmounted state according to whether or not one of the first count number and the second count number has reached a predetermined value (a fifth structure).

In addition, in the third or fourth structure described above, the mounting determination unit may change a count number if at least one of the OFF voltage signal and the ON voltage signal does not satisfy a mounting determination condition, and otherwise resets the count number while performing the mounting determination, and when the count number reaches a predetermined value, the mounting determination unit performs unmounted state determination (a sixth structure).

In addition, one of the first to sixth structures described above may further include a signal output unit arranged to perform a process of extracting an envelope based on an output signal of the transimpedance amplifier so as to output a pulse wave signal, in which the mounting determination unit may compare the pulse wave signal with a predetermined fourth threshold voltage so as to perform the mounting determination (a seventh structure).

In addition, in one of the first to seventh structures described above, the mounting determination unit may monitor the OFF voltage signal a plurality of times at a predetermined sampling rate (an eighth structure).

In addition, in the eighth structure described above, the sampling rate may be 1 to 8 Hz (a ninth structure).

In addition, in the eighth or ninth structure described above, the mounting determination unit may compare each of the OFF voltage signals monitored a plurality of times during a predetermined determination period with the first threshold voltage, so as to perform the mounting determination based on all comparison results (a tenth structure).

In addition, in the tenth structure described above, the determination period may be 1 to 5 seconds (an eleventh structure).

In addition, in one of the first to eleventh structures, the frame frequency may be 50 to 1000 Hz (a twelfth structure).

In addition, in one of the first to twelfth structures, the duty may be 1/8 to 1/200 (a thirteenth structure).

In addition, in one of the first to thirteenth structures, the mounting determination unit may output a result of the mounting determination via a general input/output port or a serial communication port (a fourteenth structure).

In addition, in one of the first to fourteenth structures, output wavelength of the light emitting unit is within a visible light range of 600 nm or less (a fifteenth structure).

In addition, a pulse wave measurement module according to another aspect of the present invention includes:

a light sensor unit arranged to irradiate a living body with light from a light emitting unit and to detect reflected light or transmitted light from the living body by a light receiving unit, so as to generate a current signal corresponding to received light intensity;

a pulse drive unit arranged to turn on and off the light emitting unit at a predetermined frame frequency and duty;

a transimpedance amplifier arranged to convert the current signal into a voltage signal;

a signal output unit arranged to perform a process of extracting an envelope based on an output signal of the transimpedance amplifier so as to output a pulse wave signal;

a generation unit arranged to generate pulse wave information based on the pulse wave signal output from the signal output unit;

a mounting determination unit arranged to perform mounting determination by comparing an OFF voltage signal obtained by the transimpedance amplifier during an OFF period of the light emitting unit with a predetermined threshold voltage;

a first transmission unit arranged to externally transmit the pulse wave information generated by the generation unit; and

a second transmission unit arranged to externally transmit a result of the determination by the mounting determination unit (a sixteenth structure).

In addition, in the sixteenth structure described above, the first transmission unit may be a serial data communication port, while the second transmission unit may be one of the serial data communication port and a general input/output port.

### Effects of the Invention

According to the present invention, it is possible to provide a pulse wave sensor that can quickly and correctly determine mounted/unmounted on a living body.

### BRIEF DESCRIPTION OF THE DRAWINGS

FIG. 1 is a schematic diagram for explaining a principle of pulse wave measurement on wrist.

FIG. 2 is a waveform chart illustrating a manner in which attenuation of light (light absorption) in a living body varies over time.

FIG. 3 is a block diagram illustrating a structural example of a pulse wave sensor.

FIG. 4 is a circuit diagram illustrating a structural example of a light sensor unit and a pulse drive unit.

FIG. 5 is a block diagram illustrating a structural example of a filter unit.

FIG. 6 is a circuit diagram illustrating a structural example of a transimpedance amplifier.

FIG. 7 is a block diagram illustrating a structural example of a control unit.

FIG. 8 is a block diagram illustrating a structural example of a pulse wave measurement module.

FIG. 9 is a block diagram illustrating a structure of the pulse wave measurement module according to a variation example.

FIG. 10 is a diagram schematically illustrating a waveform of a voltage signal Sa.

FIG. 11 is a flowchart illustrating an example of a mounting determination process.

FIG. 12 is a time chart illustrating first behaviors of signals Sa and Se (with belt fastened).

FIG. 13 is a time chart illustrating second behaviors of the signals Sa and Se (with belt unfastened).

FIG. 14 is a time chart illustrating third behaviors of the signals Sa and Se (5 mm separated).

FIG. 15 is a time chart illustrating fourth behaviors of the signals Sa and Se (left on a desk).

FIG. 16 is a time chart illustrating fifth behaviors of the signals Sa and Se (with sensor facing down).

FIG. 17 is a time chart illustrating sixth behaviors of the signals Sa and Se (with sensor facing up).

FIG. 18 is a chart illustrating an actual waveform example of the voltage signal Sa.

FIG. 19 is a chart illustrating an actual waveform example of the output signal Se.

FIG. 20 is a flowchart of a first modified example of the mounting determination process.

FIG. 21 is a flowchart specifically illustrating the process in Step S2 of FIG. 20.

FIG. 22 is a flowchart of a second modified example of the mounting determination process.

FIG. 23 is a list showing a signal measurement example in individual ambient environments.

FIG. 24 is a chart illustrating a measured signal waveform example in an indoor environment.

FIG. 25 is a chart illustrating a measured signal waveform example in an outdoor environment.

FIG. 26 is a chart illustrating a measured signal waveform example in a darkroom environment.

### BEST MODE FOR CARRYING OUT THE INVENTION

<Principle of Pulse Wave Measurement>

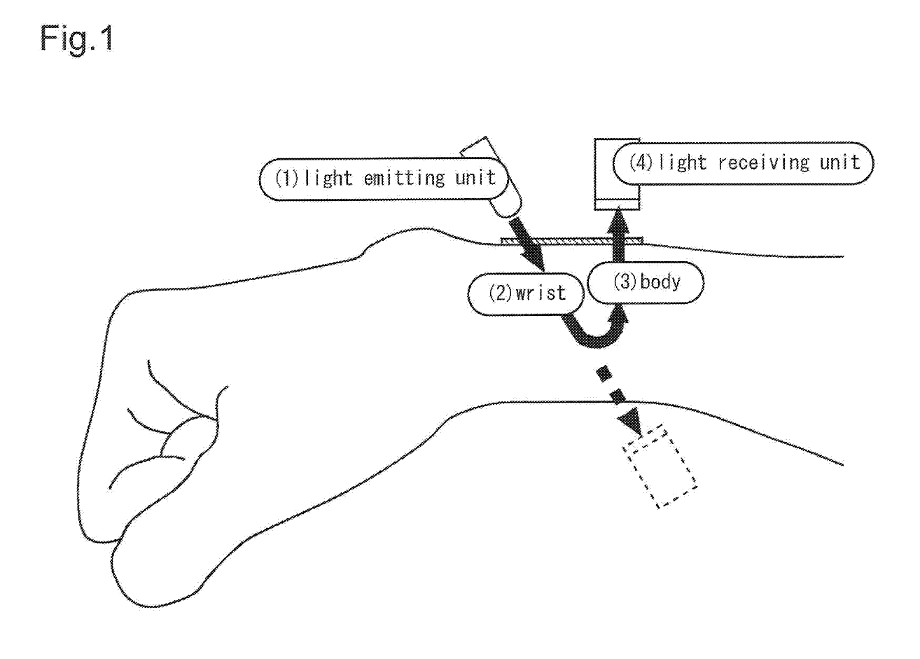

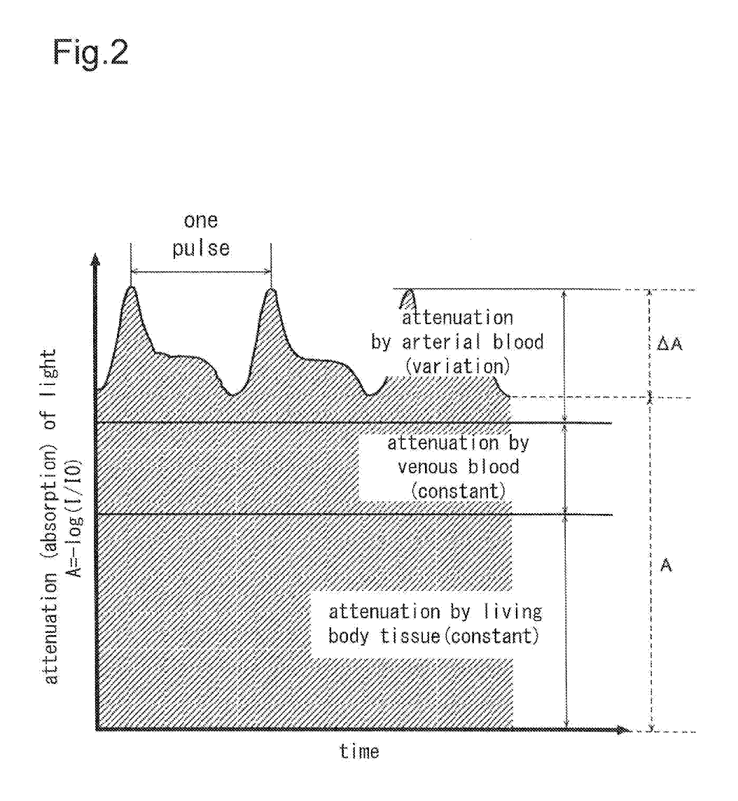

FIG. 1 is a schematic diagram for explaining a principle of pulse wave measurement on wrist, and FIG. 2 is a waveform chart illustrating a manner in which attenuation of light (light absorption) in a living body varies over time.

In pulse wave measurement using a volume pulse wave method, as illustrated in FIG. 1, for example, a light emitting unit (such as a light emitting diode (LED) emits light to a part of a living body (a wrist in FIG. 1) that is pressed to a measurement window, and intensity of light, which has passed through the body and emerges from the body, is detected by a light receiving unit (such as a photodiode or a phototransistor). Here, as illustrated in FIG. 2, attenuation of light (light absorption) by a living body tissue and venous blood (deoxyhemoglobin Hb) is constant, but attenuation of light (light absorption) by arterial blood (oxyhemoglobin HbO₂) varies over time due to beat. Therefore it is possible to measure volume pulse wave in a non-invasive manner by measuring light absorption variation of a peripheral artery using a “living body window” in a visible light region and a near infrared region (i.e. a wavelength region in which light easily passes through a living body).

Note that, for convenience sake of illustration, FIG. 1 shows a manner in which the pulse wave sensor (the light emitting unit and the light receiving unit) is mounted on a dorsal side (outer side) of a wrist, but the mount position of the pulse wave sensor is not limited to this. It may be mounted on a ventral side (inner side) of the wrist or on another part (such as a fingertip, a third joint of a finger, a forehead, a glabella, a tip of nose, a cheek, under an eye, a temple, or an earlobe).

<Information From Pulse Wave>

Note that the pulse wave controlled by a heart and autonomic nerves does not always show a constant behavior but varies (fluctuates) in various ways depending on a state of the subject. Therefore it is possible to obtain various body information of the subject by analyzing the variation (fluctuation) of the pulse wave. For example, a heart rate shows athletic ability of the subject, tension on the subject, and the like, while a heart rate fluctuation shows tiredness of the subject, good sleepiness, a stress level, and the like. In addition, an acceleration pulse wave, which is obtained by twice differentiating the volume pulse wave with respect to time, shows a vascular age, an arteriosclerosis degree and the like of the subject.

<Pulse Wave Sensor>

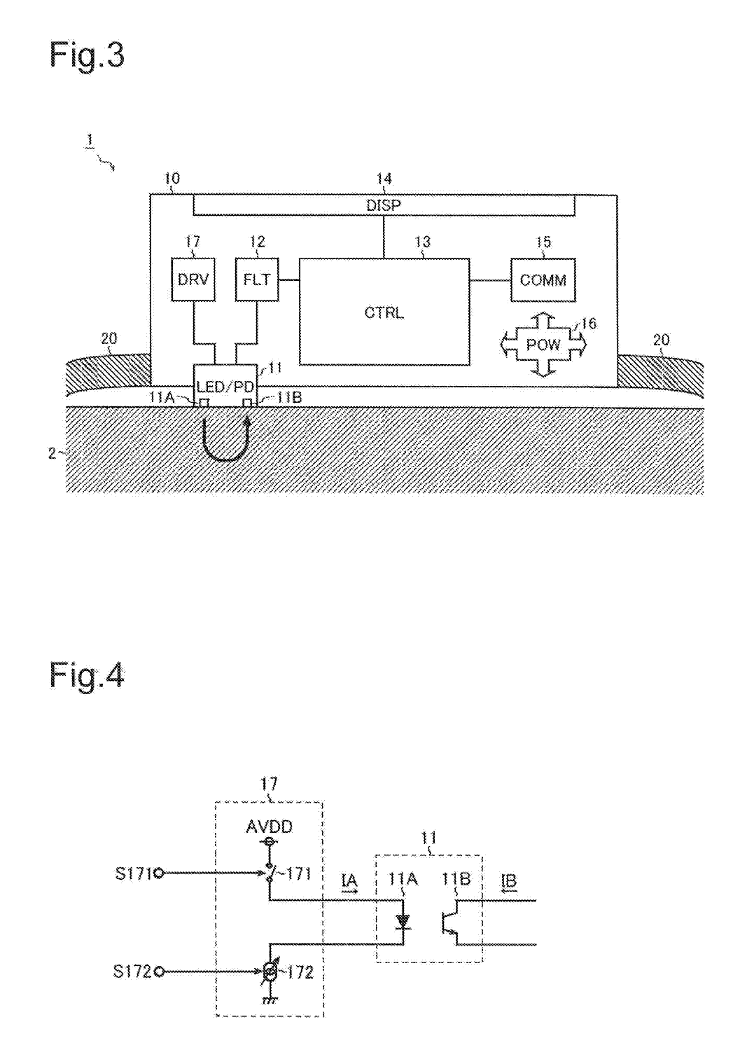

FIG. 3 is a block diagram illustrating a structural example of the pulse wave sensor. A pulse wave sensor 1 of this structural example has a wrist band structure (watch type structure) including a main body unit 10, and a belt 20 attached to both ends of the main body unit 10 so as to be put around a living body 2 (specifically a wrist). As a material of the belt 20, it is possible to use leather, metal, resin, or the like.

The main body unit 10 includes alight sensor unit 11, a filter unit 12, a control unit 13, a display unit 14, a communication unit 15, a power supply unit 16, and a pulse drive unit 17.

The light sensor unit 11 is disposed on a backside of the main body unit 10 (on the side facing the living body 2), and it irradiates the living body 2 with light from a light emitting unit 11A and detects reflected light (or transmitted light) from the living body 2 by a light receiving unit 11B, so as to generate a current signal corresponding to received light intensity. In the pulse wave sensor 1 of this structural example, the light sensor unit 11 does not have a structure in which the light emitting unit 11A and the light receiving unit 11B are disposed on opposite sides with respect to the living body 2 (so-called a transparent type as shown in FIG. 1 by a broken line arrow), but has a structure in which the light emitting unit 11A and the light receiving unit 11B are disposed on the same side with respect to the living body 2 (so-called a reflection type as shown in FIG. 1 by a solid line arrow). Note that the inventors of this application have confirmed that the pulse wave measurement on wrist is sufficiently capable of measuring pulse waves by actual experiment.

The filter unit 12 performs various signal processings (current/voltage conversion processing, detection processing, filter processing, and amplification processing) on the current signal input from the light sensor unit 11 so as to output to the control unit 13. Note that a specific structure of the filter unit 12 will be described later in detail.

The control unit 13 integrally controls the entire operation of the pulse wave sensor 1 and performs various signal processings on the output signal of the filter unit 12, so as to obtain various information about the pulse wave (a fluctuation of the pulse wave, a heart rate, a heart rate fluctuation, an acceleration pulse wave, and the like).

The display unit 14 is disposed on a surface of the main body unit 10 (a surface that does not face the living body 2), and it outputs display information (including information about date and time, a result of pulse wave measurement, and the like). In other words, the display unit 14 corresponds to a face of a watch. Note that a liquid crystal display panel or the like can be appropriately used as the display unit 14.

The communication unit 15 transmits measured data by the pulse wave sensor 1 to an external device (such as a personal computer or a cellular phone) via wireless or wired. In particular, in a structure in which the measured data by the pulse wave sensor 1 is transmitted via wireless to an external device, it is not necessary to connect the pulse wave sensor 1 and the external device with wire, and hence real time transmission of the measured data can be performed without restricting actions of the subject, for example. In addition, in order that the pulse wave sensor 1 has a waterproof structure, in view of eliminating all external terminals, it is desirable to adopt the wireless transmission method as an external transmission method of the measured data. Note that when the wireless transmission method is adopted, a Bluetooth (registered trade mark) wireless communication module IC or the like can be appropriately used as the communication unit 15.

The power supply unit 16 includes a battery and a DC/DC converter, and converts an input voltage from the battery into a desired output voltage so as to supply to individual portions of the pulse wave sensor 1. In this way, the pulse wave sensor 1 of a battery drive type does not need to connect a power supply cable externally when measuring a pulse wave, and hence it is possible to measure a pulse wave without restricting actions of the subject. Note that, as the above-mentioned battery, it is desired to use a secondary battery (such as a lithium ion secondary battery or an electrical double layer capacitor), which can be charged repeatedly. In this way, with the structure using a secondary battery as the battery, a tiresome battery exchange work is not necessary, and hence convenience of the pulse wave sensor 1 can be improved. In addition, an external power supply method when charging the battery may be a contact power supply method using a universal serial bus (USB) cable or the like, or it may be a non-contact power supply method such as an electromagnetic induction method, an electric field coupling method, or a magnetic field resonance method. However, in order that the pulse wave sensor 1 has a waterproof structure, in view of eliminating all external terminals, it is desired to adopt the non-contact power supply method as the external power supply method.

The pulse drive unit 17 turns on and off the light emitting unit 11A of the light sensor unit 11 at a predetermined frame frequency f (e.g. 50 to 1000 Hz) and duty D (1/8 to 1/200 ).

As described above, when the pulse wave sensor 1 has the wrist band structure, the pulse wave sensor 1 hardly drops off from the wrist during measurement of a pulse wave unless the subject intentionally removes the pulse wave sensor 1 from the wrist, and hence it is possible to perform measurement of the pulse wave without restricting actions of the subject.

In addition, when the pulse wave sensor 1 has the wrist band structure, the subject does not need to be conscious that the pulse wave sensor 1 is mounted, an excessive stress is not applied to the subject when performing continuous pulse wave measurement over a long period of time (a few days to a few months).

In particular, when the pulse wave sensor 1 has the display unit 14 that can display not only a result of the pulse wave measurement but also date and time information or the like (i.e., when the pulse wave sensor 1 has a watch type structure), the subject can put on the pulse wave sensor 1 as a watch on a daily basis, and hence reluctance to wear the pulse wave sensor 1 can be wiped out, so that it is possible to contribute to creation of a new user layer.

In addition, it is desired that the pulse wave sensor 1 should haves a waterproof structure. With this structure, even if the pulse wave sensor 1 is drenched in water (rain) or sweat, it can measure a pulse wave without being broken down. In addition, when the pulse wave sensor 1 is shared by multiple users (e.g. used as a rental in a sports gym), the pulse wave sensor 1 can be washed with water so that the pulse wave sensor 1 can be maintained to be clean.

<Light Sensor Unit and Pulse Drive Unit>

FIG. 4 is a circuit diagram illustrating a structural example of the light sensor unit 11 and the pulse drive unit 17. The light sensor unit 11 of this structural example includes the light emitting diode (corresponding to the light emitting unit) 11A and the phototransistor (corresponding to the light receiving unit) 11B. In addition, the pulse drive unit 17 of this structural example includes a switch 171 and a current source 172.

An anode of the light emitting diode 11A is connected to an application terminal of a power supply voltage AVDD via the switch 171. A cathode of the light emitting diode 11A is connected to a ground terminal via the current source 172. The switch 171 is turned on and off according to a pulse drive signal S171. The current source 172 generates a constant current IA corresponding to a luminance control signal S172. Note that it is desired to pulse-drive the light emitting diode 11A at a luminance higher than extraneous light in order to perform accurate pulse wave measurement during exercise or outdoors.

When the switch 171 is turned on, a current path for the constant current IA to flow is formed, and hence the light emitting diode 11A is turned on and emits light to irradiate the living body 2. In this case, a current signal IB corresponding to received light intensity of the reflected light from the living body 2 flows between a collector and an emitter of the phototransistor 11B. On the other hand, when the switch 171 is turned off, the current path for the constant current IA is broken, and hence the light emitting diode 11A is turned off.

<Filter Unit>

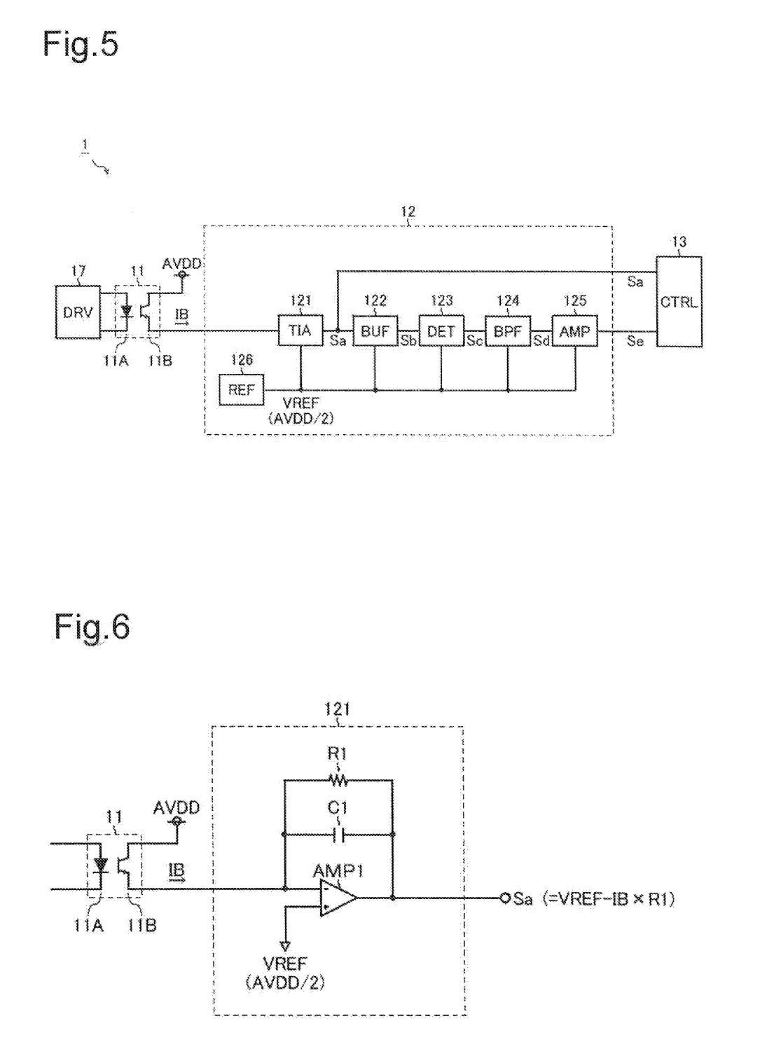

FIG. 5 is a block diagram of a structural example of the filter unit 12. The filter unit 12 of this structural example includes a transimpedance amplifier 121 (hereinafter referred to as a transimpedance amplifier (TIA) 121), a buffer circuit 122, a detector circuit 123, a band-pass filter circuit 124, an amplifier circuit 125, and a reference voltage generation circuit 126. Note that the structure from the buffer circuit 122 to the amplifier circuit 125 on the post-stage of TIA 121 forms a signal output unit that outputs an output signal Se described later (corresponding to the pulse wave signal).

The TIA 121 is one type of current/voltage conversion circuit that converts the current signal IB into a voltage signal Sa so as to output to each of the buffer circuit 122 and the control unit 13 on the post-stage.

The buffer circuit 122 is a voltage follower that transfers the voltage signal Sa as a buffer signal Sb to the post-stage.

The detector circuit 123 extracts an envelope of the voltage signal Sb of pulse drive so as to generate a detection signal Sc, which is output to the post-stage. A half-wave rectification detector circuit, a full-wave rectification detector circuit, or the like can be used as the detector circuit 123.

The band-pass filter circuit 124 removes both low frequency components and high frequency components superimposed on the detection signal Sc so as to generate a filter signal Sd, which is output to the post-stage. Note that it is desired to set a pass frequency band of the band-pass filter circuit 124 to approximately 0.6 to 4.0 Hz.

The amplifier circuit 125 generates the output signal Se by amplifying the filter signal Sd with a predetermined gain, so as to outputs it to the control unit 13 of the post-stage.

The reference voltage generation circuit 126 divides the power supply voltage AVDD by 1/2 so as to generate a reference voltage VREF (=AVDD/2), which is supplied to each portion of the filter unit 12.

The filter unit 12 according to this structural example can appropriately eliminate body motion noise of the subject, and hence it can accurately detect not only the pulse wave when the subject is resting but also the pulse wave when the subject is exercising (walking, jogging, or running).

In addition, in the filter unit 12 of this structural example, each of the TIA 121, the buffer circuit 122, the detector circuit 123, the band-pass filter circuit 124, and the amplifier circuit 125 operates with respect to the reference voltage VREF (=AVDD/2) as a center, and hence the output signal Se of the filter unit 12 has a waveform in which the amplitude varies up and down with respect to the reference voltage VREF. Therefore the filter unit 12 of this structural example can correctly detect the pulse wave data by preventing saturation of the output signal Se (to the power supply voltage AVDD or to the ground voltage GND).

<TIA>

FIG. 6 is a circuit diagram illustrating a structural example of the TIA 121. The TIA 121 of this structural example includes an operational amplifier AMP1, a resistor R1, and a capacitor C1. A non-inverting input terminal (+) of the operational amplifier AMP1 is connected to an application terminal of the reference voltage VREF (=AVDD/2). An inverting input terminal (−) of the operational amplifier AMP1 is connected to an emitter of the photodiode 11B. A collector of the photodiode 11B is connected to an application terminal of the power supply voltage AVDD. An output terminal of the operational amplifier AMP1 corresponds to an output terminal of the voltage signal Sa. The resistor R1 and the capacitor C1 are connected in parallel between the inverting input terminal (−) of the operational amplifier AMP1 and the output terminal.

In the TIA 121 of this structural example, the current signal IB flows in the current path from the inverting input terminal (−) of the operational amplifier AMP1 via the resistor R1 to the output terminal of the voltage signal Sa. Therefore the inverting input terminal (−) of the operational amplifier AMP1 is applied with a voltage (=Sa+IB×R1) obtained by adding a voltage across terminals of the resistor R1 to the voltage signal Sa. On the other hand, the operational amplifier AMP1 generates the output signal Sa so that the non-inverting input terminal (+) and the inverting input terminal (−) are imaginarily short-circuited. Therefore the voltage signal Sa generated by the TIA 121 has a voltage value (VREF−IB×R1) obtained by subtracting the voltage across terminals of the resistor R1 from the reference voltage VREF.

In other words, as the current signal IB flowing in the resistor R1 (corresponding to light amount received by the phototransistor 11B) is higher, the voltage signal Sa becomes lower. On the contrary, as the current signal IB is lower, the voltage signal Sa becomes higher. Note that a gain of the TIA 121 can be arbitrarily adjusted by changing a resistance of the resistor R1.

<About Control Unit>

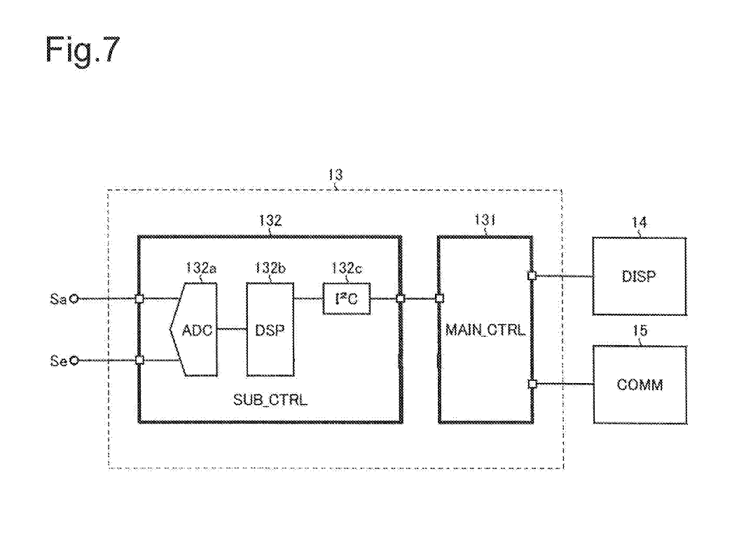

FIG. 7 is a block diagram illustrating a structural example of the control unit 13. The control unit 13 of this structural example includes a main control circuit 131 and a sub-control circuit 132.

The main control circuit 131 mainly controls display operation using the display unit 14 and communication operation using the communication unit 15.

The sub-control circuit 132 mainly controls pulse wave measurement operation using the light sensor unit 11, and it includes an A/D converter 132a, a digital signal processing unit 132b, and a serial data communication port 132c. Note that the pulse wave measurement operation described above includes, for example, pulse drive control and luminance setting control (calibration) of the light emitting unit 11A, digital signal processing of the output signal Se, and a mounting determination process based on the voltage signal Sa and the output signal Se.

The A/D converter 132a receives the output signal Se of an analog format and the voltage signal Sa in a time sharing manner, and it converts each of them into a digital format and sequentially outputs them to the digital signal processing unit 132b. Note that a plurality of single input type A/D converters may be disposed in parallel for separately receiving the output signal Se and the voltage signal Sa, instead of the multiple input type A/D converter 132a.

The digital signal processing unit 132b performs various digital signal processings on an output of the A/D converter 132a. The digital signal processings include a waveform shaping process and an analyzing process of the pulse wave data based on the output signal Se, and the mounting determination process based on the voltage signal Sa and the output signal Se. In other words, the digital signal processing unit 132b has a function as a mounting determination unit for determining a mounted/unmounted state of the pulse wave sensor 1. Details of the mounting determination process will be described later. In addition, the analyzing process includes a process of calculating and generating various information about the pulse wave (such as the heart rate, the heart rate fluctuation, the acceleration pulse wave, and the like).

The serial data communication port 132c is a port for performing serial data communication between the main control circuit 131 and the sub-control circuit 132. For example, the digital signal processing unit 132b transmits the various information about the pulse wave (pulse wave information) obtained by the pulse wave measurement operation to the main control circuit 131 via the serial data communication port 132c. The main control circuit 131 controls the display unit 14 to display the pulse wave information transmitted from the sub-control circuit 132 and controls the communication unit 15 to transmit the same to an external device.

In addition, the digital signal processing unit 132b can also transmit a mounting determination result of the pulse wave sensor 1 to the main control circuit 131 via the serial data communication port 132c. For example, the main control circuit 131 regularly transmits a request signal via the serial data communication port 132c, and the digital signal processing unit 132b receives the request signal and replies the mounting determination result of the pulse wave sensor 1 via the serial data communication port 132c.

Note that an I²C port or the like can be appropriately used as the serial data communication port 132c.

<About Pulse Wave Measurement Module>

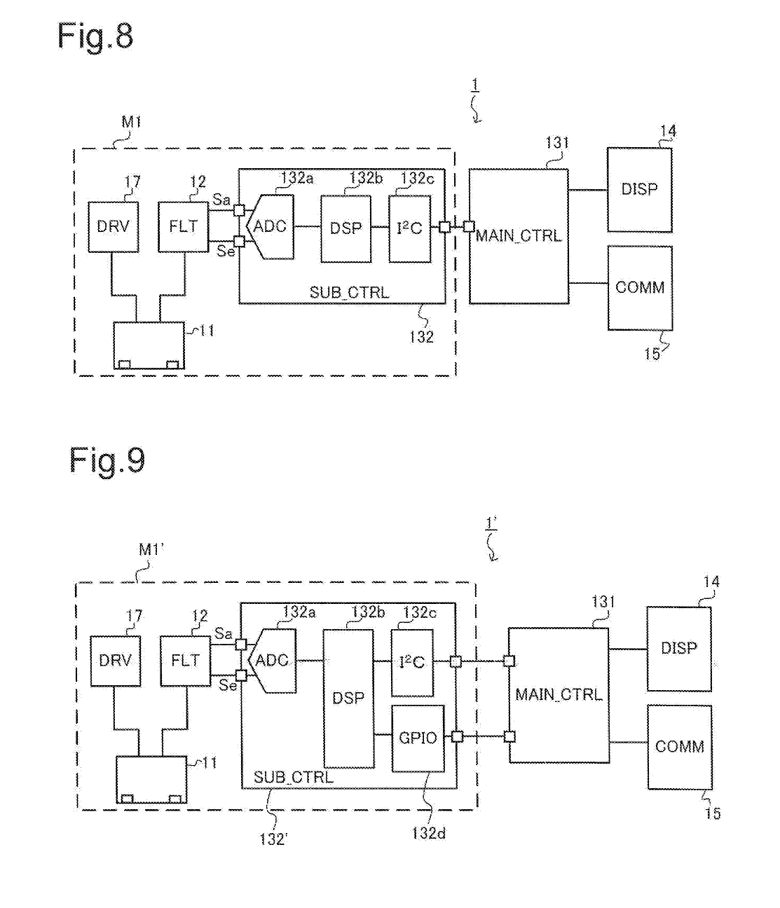

In the pulse wave sensor 1 according to this embodiment, as illustrated in FIG. 8, the light sensor unit 11, the filter unit 12, the pulse drive unit 17, and the sub-control circuit 132 are modularized as a pulse wave measurement module M1.

The digital signal processing unit 132b included in the sub-control circuit 132 of the pulse wave measurement module M1 performs a pulse wave information generation process and the mounting determination process, and results of the processes are transmitted to the main control circuit 131 via the serial data communication port 132c. Thus, the main control circuit 131 is not required to perform the above-mentioned processes and hence the load can be assigned to other controls. Note that throughput of the digital signal processing unit 132b may lower than the main control circuit 131.

In addition, FIG. 9 illustrates a structure of a pulse wave sensor 1′ according to a variation example. In FIG. 9, as a different point from FIG. 8, a sub-control circuit 132′ includes a general input/output port 132d in addition to the serial data communication port 132c. Further, the light sensor unit 11, the filter unit 12, the pulse drive unit 17, and the sub-control circuit 132′ are modularized as a pulse wave measurement module M1′.

The general input/output port 132d is a port for performing input/output of a one-bit signal (binary signal). For example, the digital signal processing unit 132b outputs a mounting determination flag (corresponding to the mounting determination result) to the general input/output port 132d. Specifically, the digital signal processing unit 132b sets the general input/output port 132d to a high level when determining that the pulse wave sensor 1 is correctly mounted, and it sets the general input/output port 132d to a low level when determining that the pulse wave sensor 1 is not correctly mounted. The main control circuit 131 monitors output logic level of the general input/output port 132d and controls the display unit 14 to display the monitored result or controls the communication unit 15 to transmit the same to the external device. Note that a general purpose input/output (GPIO) port or the like can be appropriately used as the general input/output port 132d.

<Mounting Determination Process>

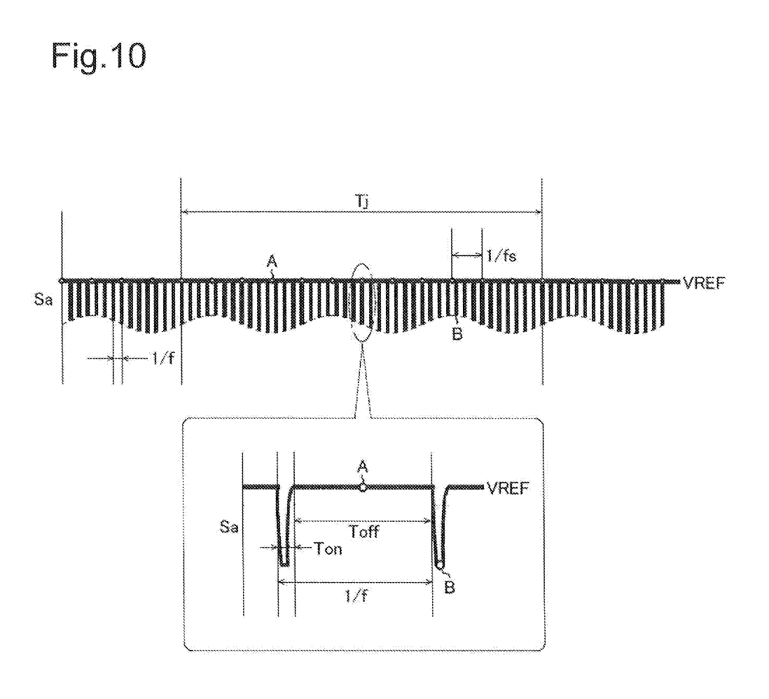

FIG. 10 shows a signal waveform of the voltage signal Sa and a partial enlarged diagram thereof in a state where the pulse wave sensor 1 is correctly mounted. As described above, the voltage signal Sa generated by the TIA 121 has the voltage value (VREF−IB×R1) obtained by subtracting the voltage across terminals of the resistor R1 from the reference voltage VREF. Here, the received light intensity of the light receiving unit 11B (therefore a current value of the current signal IB) in an ON period Ton of the light emitting unit 11A varies along with a beat of the subject. Thus, as shown in the chart by a point B, the voltage signal Sa (an ON voltage signal Sa@B) obtained by the TIA 121 during the ON period Ton of the light emitting unit 11A is envelope-detected, and hence the pulse wave data of the subject (see a thin broken line in the chart) can be obtained.

On the other hand, if no light enters the light receiving unit 11B so that no current signal IB flows in the resistor R1, the voltage signal Sa is ideally identical to the reference voltage VREF. For example, in a state where a light sensor 1 is correctly mounted on the living body 2 (in a state where extraneous light is appropriately prevented from entering the light receiving unit 11B), the received light intensity of the light receiving unit 11B in an OFF period Toff of the light emitting unit 11A is substantially zero, and hence the current signal IB hardly flows in the resistor R1. Thus, as shown in the chart by a point A, the voltage signal Sa obtained by the TIA 121 during the OFF period Toff of the light emitting unit 11A (an OFF voltage signal Sa@A) must be substantially identical to the reference voltage VREF.

In view of the above-mentioned finding, the control unit 13 (particularly the digital signal processing unit 132b) has a structure for comparing the OFF voltage signal Sa@A with a predetermined threshold voltage Vth so as to perform the mounting determination process of the pulse wave sensor 1.

Note that, when the mounting determination process of the pulse wave sensor 1 described below is performed, it is desired that the frame frequency f in the pulse drive of the light emitting unit 11A should be set to a value within the range from 50 to 1000 Hz (e.g. f=128 Hz). In addition, it is desired that the duty D (a ratio of the ON period Ton to the frame period) in the pulse drive of the light emitting unit 11A should be set to a value within the range from 1/8 to 1/200 (e.g. D=1/16).

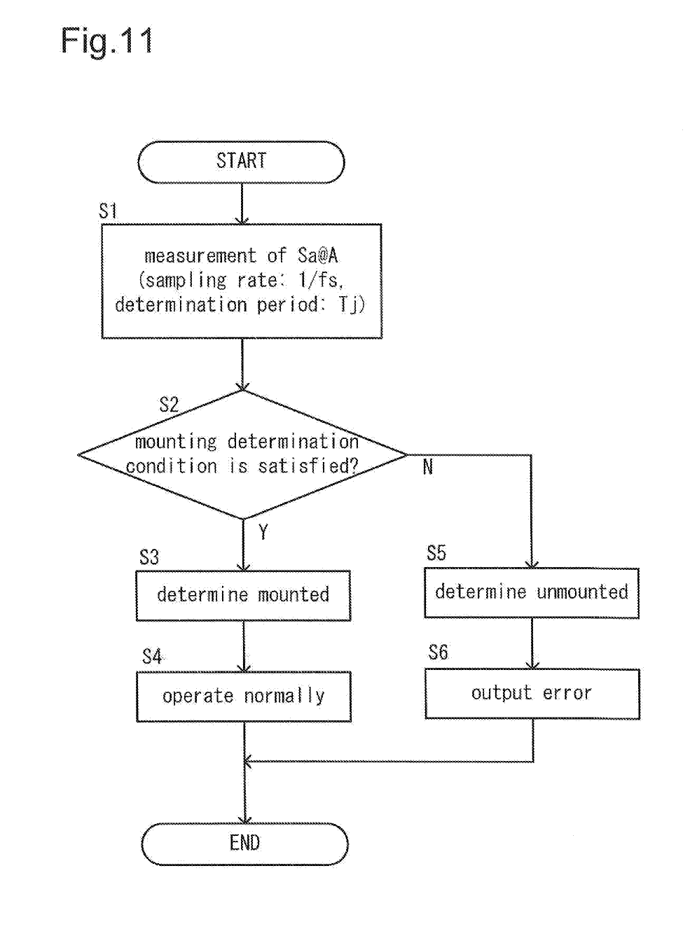

FIG. 11 is a flowchart illustrating an example of the mounting determination process. When the mounting determination process is started, first in Step S1, measurement of the OFF voltage signal Sa@A (monitoring a plurality of times) is performed at a predetermined sampling rate fs (e.g. fs=1 to 8 Hz) during a predetermined determination period Tj (e.g. Tj=1 to 5 seconds). For example, if the determination period Tj is 3 seconds and the sampling rate fs is 4 Hz, the measurement is performed total 12 times (i.e. 3 seconds×4 Hz) in Step S1.

In the next Step S2, each of the OFF voltage signals Sa@A monitored a plurality of times during the determination period Tj is compared with the predetermined threshold voltage Vth, and it is determined whether or not a predetermined mounting determination condition is satisfied based on all comparison results. Here, if the determination is positive, the flow proceeds to Step S3. If the determination is negative, the flow proceeds to Step S5.

Note that the threshold voltage Vth is set to a voltage value lower than the reference voltage VREF of the TIA 121. For example, if the reference voltage VREF is 1.50 V, it is desired to set the threshold voltage Vth to a value within the range from 1.40 to 1.49 V (e.g. Vth=1.49 V). As described above, in a state where the light sensor 1 is correctly mounted on the living body 2, the received light intensity of the light receiving unit 11B in the OFF period Toff of the light emitting unit 11A is substantially zero, and hence the OFF voltage signal Sa@A must be higher than the threshold voltage Vth.

Therefore it is possible to determine whether or not the pulse wave sensor 1 is correctly mounted on the living body 2 by comparing each of monitored OFF voltages Sa@A with the threshold voltage Vth so as to verify the comparison result against the predetermined mounting determination condition.

Note that the mounting determination condition is: (1) all the monitored OFF voltages Sa@A are higher than the threshold voltage Vth; (2) substantially all (80 to 90%) of them are higher than the threshold voltage Vth; (3) more than a half of them are higher than the threshold voltage Vth; or the like. Among these example conditions, (1) is the most severe condition, while (3) is the least severe condition, as a matter of course.

If the determination is positive in Step S2, it is determined in Step S3 that the pulse wave sensor 1 is correctly mounted on the living body 2. Then, in the next Step S4, the process proceeds to normal operation, and the series of mounting determination flow is finished.

On the other hand, if the determination is negative in Step S2, it is determined in Step S5 that the pulse wave sensor 1 is not correctly mounted on the living body 2. Then, in the next Step S5, error output (error notification to the subject) is performed using the display unit 14 or the like, and the series of mounting determination flow is finished.

In this way, with the structure in which the mounting determination of the pulse wave sensor 1 is performed based on the received light intensity of the light receiving unit 11B when the pulse-driven light emitting unit 11A is turned off, instead of reading an amplitude level of the pulse wave signal, it is possible to quickly and correctly determine mounted/unmounted on the living body 2.

In addition, in the state where the pulse wave sensor 1 is not correctly mounted on the living body 2, error output can be performed using the display unit 14 or the like, and hence it is possible to urge correct mounting on the subject.

Note that, in order to improve stability of the pulse wave measurement and accuracy of parameter calculation, it is desired to regularly repeat the series of mounting determination process during the pulse wave measurement, too.

In addition, when performing the luminance adjustment process (calibration process) of the light emitting unit 11A, it is desired to first perform the mounting determination process described above and to start the luminance adjustment process after it is confirmed that the pulse wave sensor 1 is correctly mounted on the living body 2.

<Example of Mounting Determination>

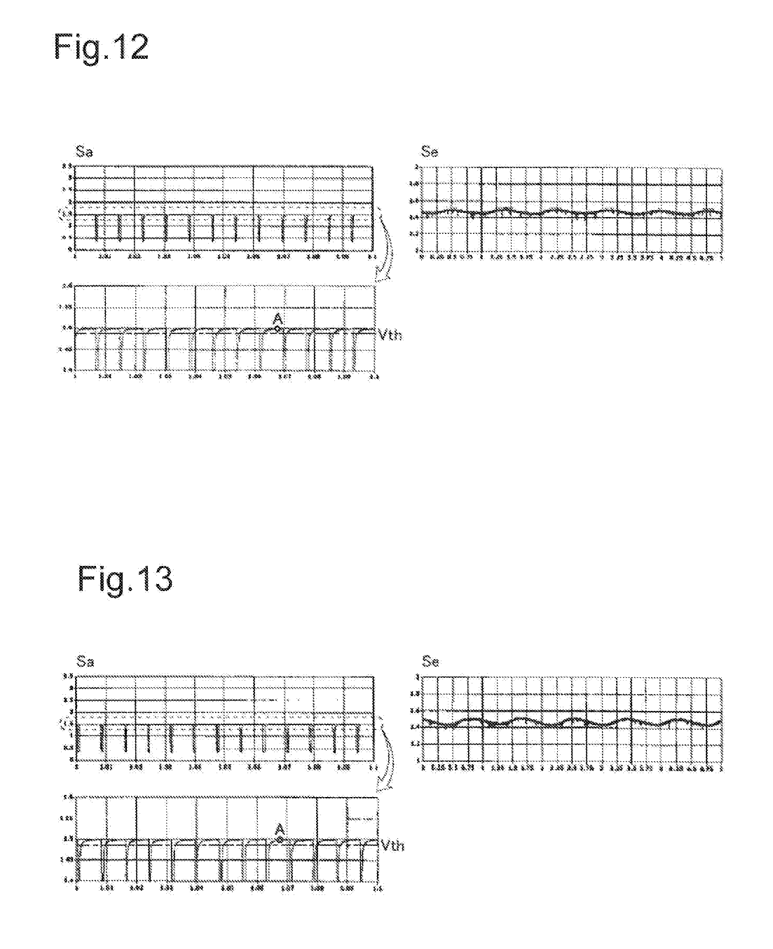

FIG. 12 is a time chart of first behaviors of the voltage signal Sa and the output signal Se (signal waveforms obtained under the condition where the pulse wave sensor 1 is fastened to the living body 2 with the belt 20). Note that, as to the voltage signal Sa, a partial enlarged chart thereof in a vicinity of the reference voltage VREF (1.5 V) is also shown. In the first behavior in this chart, the OFF voltage signal Sa@A is substantially identical to the reference voltage VREF and is higher than the threshold voltage Vth. Therefore it is determined that the pulse wave sensor 1 is correctly mounted on the living body 2.

FIG. 13 is a time chart showing second behaviors of the voltage signal Sa and the output signal Se (signal waveforms obtained under the condition where the pulse wave sensor 1 is just placed on the living body 2 but is not fastened by the belt 20). Note that, as to the voltage signal Sa, a partial enlarged chart thereof in a vicinity of the reference voltage VREF (1.5) is also shown. In the second behavior in this chart, similarly to the first behavior (FIG. 10) described above, the OFF voltage signal Sa@A is substantially identical to the reference voltage VREF and is higher than the threshold voltage Vth. Therefore it is determined that the pulse wave sensor 1 is correctly mounted on the living body 2.

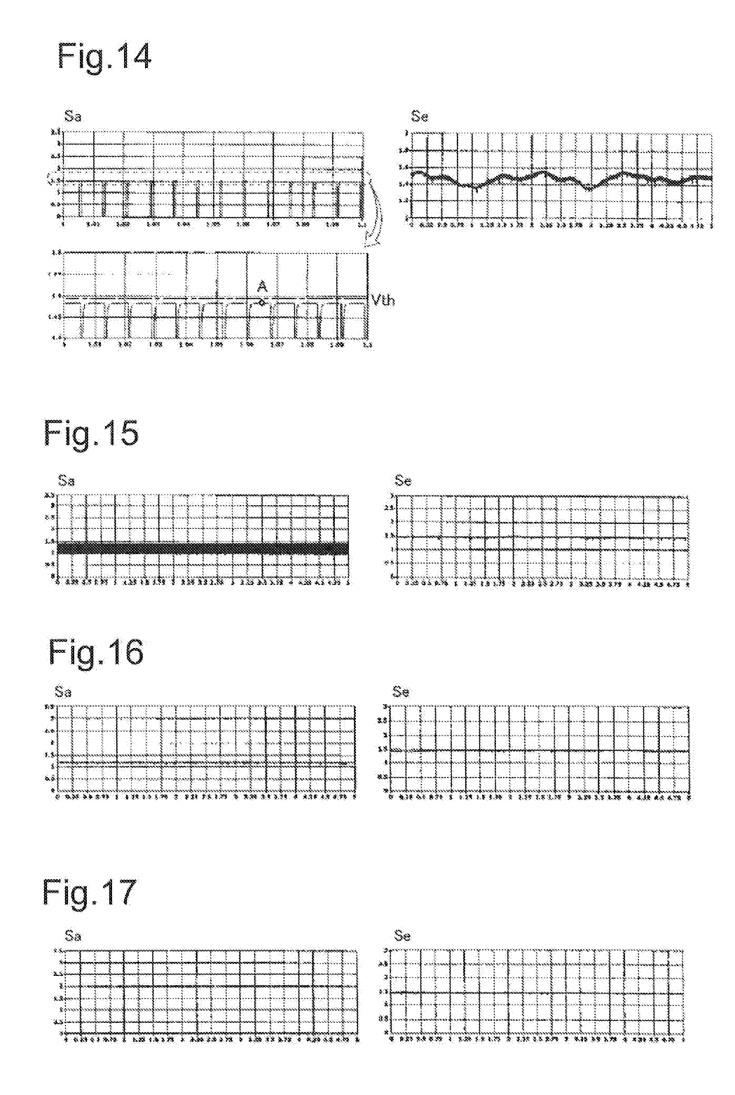

FIG. 14 is a time chart showing third behaviors of the voltage signal Sa and the output signal Se (signal waveforms obtained under the condition where a light receiving surface of the pulse wave sensor 1 is separated from the living body by 2 to 5 mm). Note that, as to the voltage signal Sa, a partial enlarged chart thereof in a vicinity of the reference voltage VREF (1.5 V) is also shown. In the third behavior in this chart, because extraneous light slightly enters the light receiving unit 11B, the OFF voltage signal Sa@A is lower than the threshold voltage Vth (1.49 V). Therefore it is determined that the pulse wave sensor 1 is not correctly mounted on the living body 2.

FIG. 15 is a time chart showing fourth behaviors of the voltage signal Sa and the output signal Se (signal waveforms obtained under the condition where the pulse wave sensor 1 is left on a desk with the light receiving surface down). In the fourth behavior in this chart, pulses of the voltage signal Sa along with turning on and off of the light emitting unit 11A cannot be discriminated. In addition, the OFF voltage signal Sa@A is apparently lower than the threshold voltage Vth without showing the partial enlarged diagram in a vicinity of the reference voltage VREF (1.5 V). Therefore it is determined that the pulse wave sensor 1 is not correctly mounted on the living body 2.

FIG. 16 is a time chart showing fifth behaviors of the voltage signal Sa and the output signal Se (signal waveforms obtained under the condition where the pulse wave sensor 1 is left in a light environment of 800 1× with the receiving surface down). In the fifth behavior in this chart, it is apparent that the voltage signal Sa is always lower than the threshold voltage Vth. Therefore it is determined that the pulse wave sensor 1 is not correctly mounted on the living body 2.

FIG. 17 is a time chart showing sixth behaviors of the voltage signal Sa and the output signal Se (signal waveforms obtained under the condition where the pulse wave sensor 1 is left in a light environment of 800 1× with the light receiving surface up). In the sixth behavior in this chart, it is apparent that the voltage signal Sa is stuck to substantially 0 v. Therefore it is determined that the pulse wave sensor 1 is not correctly mounted on the living body 2.

<Modified Example of Mounting Determination Process>

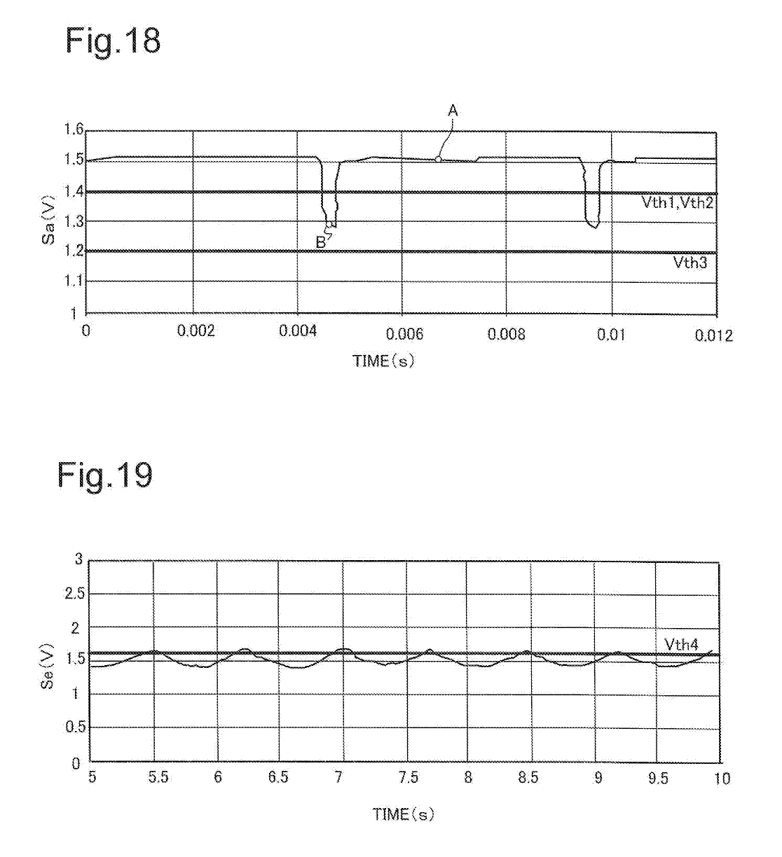

Here, FIG. 18 shows an actual waveform example of the voltage signal Sa in the state where the pulse wave sensor 1 is correctly mounted (drive condition of the light emitting unit 11A is that the frame frequency f is 200 Hz and the duty D is 1/16). As described above, the OFF voltage signal Sa@A is substantially the reference voltage VREF, i.e. 1.5 V and is constant. Therefore it is possible to perform the mounting determination by comparing the OFF voltage signal Sa@A with a first threshold voltage Vth1 (e.g. 1.4 V) that is lower than the reference voltage VREF.

In addition, in the pulse wave sensor 1, the luminance setting control (calibration) of the light emitting unit 11A is performed before starting the pulse wave measurement. The luminance setting control is performed mainly by the digital signal processing unit 132b (i.e., the digital signal processing unit 132b corresponds to the luminance adjustment control unit). For example, in a state where the current value of the current source 172 is set by the luminance control signal S172 (FIG. 4), the pulse drive signal S171 turns on and off the switch 171 for a few frames, a statistic value (e.g. an average value) of the ON voltage signal Sa@B is calculated, and the statistic value is compared with a predetermined threshold voltage (the threshold voltage for adjustment). If the statistic value is higher than the threshold voltage, it is set so that the current value is increased, and further switching of the switch 171 is performed. If the current value is increased, luminance of the light emitting unit 11A is increased so that the current value of the current signal IB is increased. Therefore the ON voltage signal Sa@B is decreased. Further, if the statistic value becomes the threshold voltage or lower, the current value at that time is set as a use current value (i.e., the luminance of the light emitting unit 11A is set). After that, the pulse drive of the light emitting unit 11A is started using the use current value, and output of the output signal Se is started (i.e., the pulse wave measurement is started).

FIG. 18 illustrates the ON voltage signal Sa@B when the threshold voltage used in the luminance setting control is 1.3 V. As illustrated in FIG. 18, when the pulse wave sensor 1 is correctly mounted, the ON voltage signal Sa@B must be within the range between a second threshold voltage Vth2 (e.g. 1.4 V) higher than a reference value that is the above-mentioned threshold voltage and a third threshold voltage Vth3 (e.g. 1.2 V) lower than the reference value. Therefore it is possible to perform the mounting determination by comparing the ON voltage signal Sa@B with the range defined by the second threshold voltage Vth2 and the third threshold voltage Vth3.

In addition, FIG. 19 illustrates an actual waveform example of the output signal Se in the state where the pulse wave sensor 1 is correctly mounted. If it is correctly mounted in this way, the output signal Se as the pulse wave signal has a waveform that vibrates between the positive side and the negative side with respect to the reference voltage VREF (=1.5 V). In this case, as shown in FIG. 19, when a voltage value higher than the reference voltage VREF is a fourth threshold voltage Vth4 (e.g. 1.6 V), there is timing when the output signal Se becomes the fourth threshold voltage Vth4 or higher. Therefore it is possible to perform the mounting determination by comparing the output signal Se with the fourth threshold voltage Vth4.

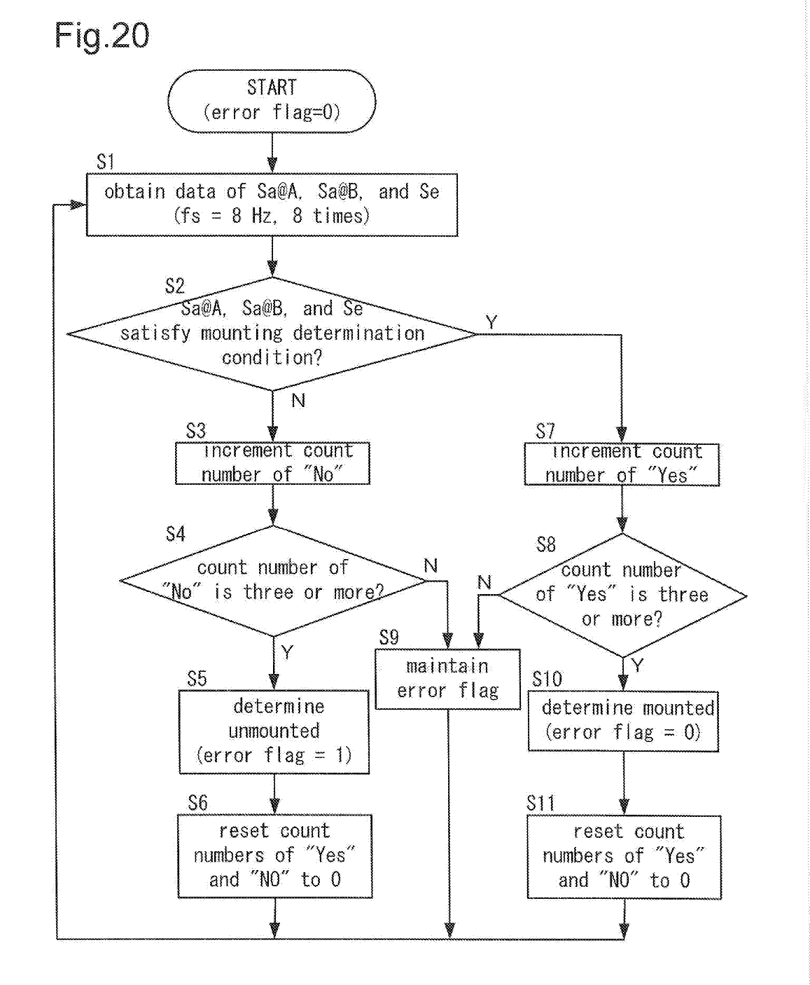

There is described below a specific mounting determination process based on a principle of the mounting determination based on the voltage signal Sa and the output signal Se. FIG. 20 is a flowchart according to a first modified example of the mounting determination process.

When a pulse wave measurement start operation (e.g. a key-press operation) is made to an operation portion (not shown in FIG. 3) in the state where the pulse wave sensor 1 is normally mounted on the living body 2, the main control circuit 131 detects the operation and controls the sub-control circuit 132 to start the pulse wave measurement operation. The sub-control circuit 132 performs the luminance setting control of the light emitting unit 11A described above and controls to start the pulse drive of the light emitting unit 11A, and hence the output of the output signal Se is started (i.e., the pulse wave measurement is started).

In this case, the flow of the mounting determination process illustrated in FIG. 20 is also started. The flow is performed mainly by the digital signal processing unit 132b. In addition, when the flow is started, the error flag (error flag) is initialized to zero.

First, in Step S1, predetermined numbers of data of the OFF voltage signal Sa@A, the ON voltage signal Sa@B, and the output signal Se are respectively obtained at a predetermined sampling frequency fs. For example, if the sampling frequency fs is 8 Hz, eight data are obtained (in this case, data are obtained for one second).

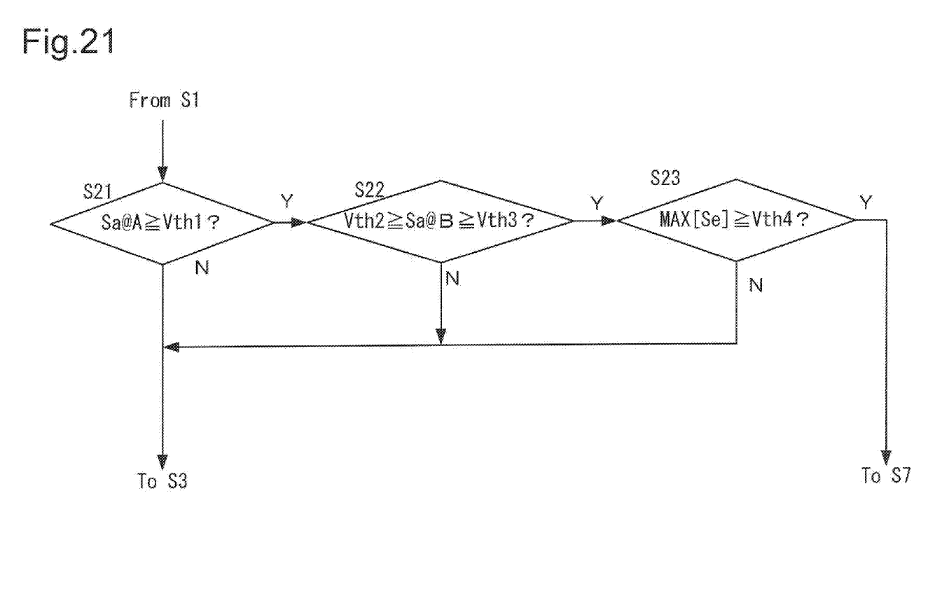

Then in Step S2, it is determined whether or not the obtained OFF voltage signal Sa@A, ON voltage signal Sa@B, and output signal Se are all satisfy the mounting determination condition. Amore specific process of Step S2 is illustrated in the flowchart of FIG. 21.

As illustrated in FIG. 21, first in Step S21, it is determined whether or not all the obtained OFF voltage signals Sa@A are the first threshold voltage Vth1 or higher. If it is true (Y in Step S21), the process proceeds to Step S22. In Step S22, all the obtained ON voltage signals Sa@B are within the range defined by the third threshold voltage Vth3 or higher and the second threshold voltage Vth2 or lower. If it is true (Y in Step S22), the process proceeds to Step S23.

In Step S23, it is determined whether or not the maximum value of the obtained output signals Se is the fourth threshold voltage Vth4 or higher. If it is true, it is determined in Step S2 (FIG. 20) that the mounting determination condition is satisfied (Y in Step S2), and the process proceeds to Step S7. On the other hand, if the condition is not satisfied in one of Steps S21, S22 and S23 (N in Steps S21, S22 or S23), it is determined in Step S2 (FIG. 20) that the mounting determination condition is not satisfied (N in Step S2), and the process proceeds to Step S3.

Note that the determination in Steps S21 and S22 whether or not the condition is satisfied may be performed based on whether or not majority (e.g. 80% or higher) or more than a half of the obtained data satisfies the condition, for example.

When proceeding to Step S3, the count number of “No” (having initial value of zero) is incremented by one, and the process proceeds to Step S4. In Step S4, it is determined whether or not the count number of “No” is a predetermined value (e.g. 3) or more. If it is false (N in Step S4), the process proceeds to Step S9 in which the error flag is maintained. In addition, when proceeding to Step S7, the count number of “Yes” (having initial value of zero) is incremented by one, and the process proceeds to Step S8. In Step S8, it is determined whether or not the count number of “Yes” is a predetermined value (e.g. 3) or more. If it is false (N in Step S8), the process proceeds to Step S9 in which the error flag is maintained. After Step S9, the process returns to Step S1.

Further in Step S4, if the count number of “No” is the predetermined value or more (Y in Step S4), the process proceeds to Step S5 in which the error flag is set to one as being unmounted (including abnormally mounted). Then, the process proceeds to Step S6 in which the count number of “Yes” and the count number of “No” are reset to zero, and the process returns to Step S1.

Further in Step S8, if the count number of “Yes” is the predetermined value or more (Y in Step S8), the process proceeds to Step S10 in which the error flag is set to zero as being correctly mounted. Then, the process proceeds to Step S11 in which the count number of “Yes” and the count number of “No” are reset to zero, and the process returns to Step S1.

For example, if the sampling frequency fs of data in Step 51 is 8 Hz, the number of the obtained data is eight, and the predetermined value as the threshold value for determination in Steps S4 and S8 is three, then the determination of mounted/unmounted state can be performed in three minutes in the shortest (=1/8×8×3). In addition, in the process illustrated in FIG. 20, even if it is determined in Step S2 that the mounting determination condition is satisfied for a certain reason despite of being unmounted actually, the count number of “No” reaches the predetermined value first, and hence the unmounted state is determined finally.

Further, if the unmounted state is determined so that the error flag is set 1, and the error flag is transmitted from the digital signal processing unit 132b to the main control circuit 131 due to the request signal from the main control circuit 131, then the main control circuit 131 instructs the sub-control circuit 132 to stop the pulse wave measurement, for example. Thus, it is possible to avoid an unnatural situation such as a display of the pulse wave information (such as the heart rate) despite of the unmounted state.

In addition, in this case, the main control circuit 131 may control the display unit 14 to make a warning display. The warning display may urge the user to mount correctly, for example. Thus, the user can be notified that the pulse wave sensor 1 is mounted but is coming off, for example. Alternatively, it is possible to notify using an LED or a speaker instead of the display unit 14, for example.

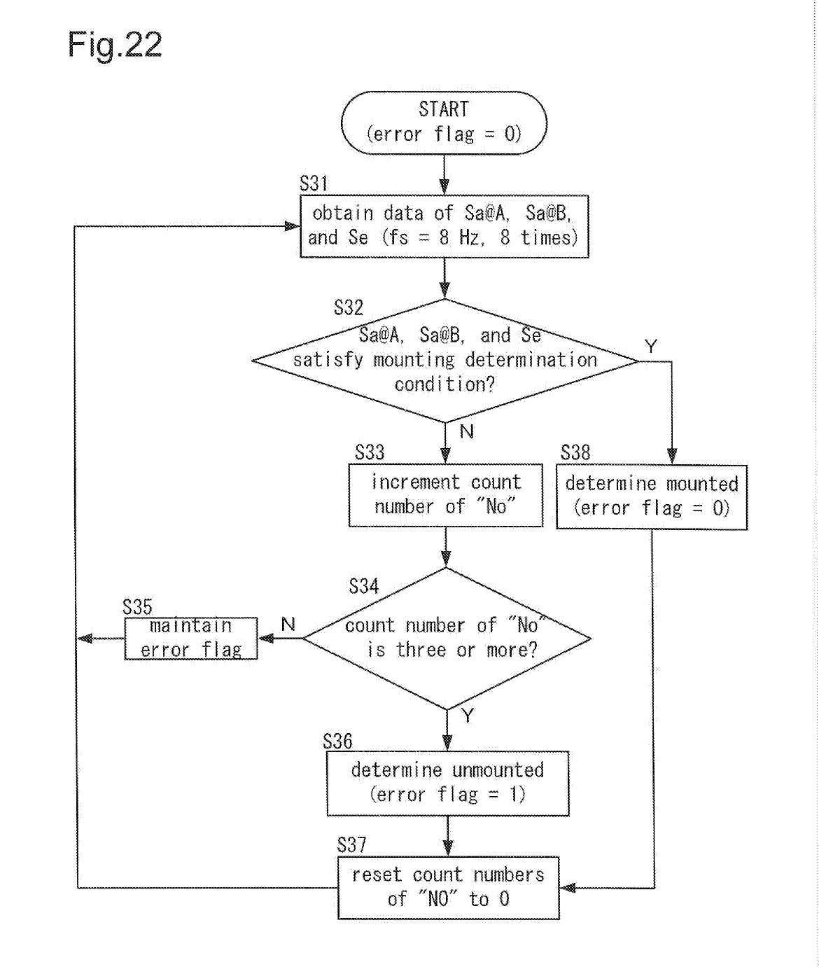

FIG. 22 illustrates a flowchart according to a second modified example of the mounting determination process. Steps S31 and S32 in the flow illustrated in this chart respectively correspond to Steps S1 and S2 in the first modified example (FIG. 20) described above, and the different points are in the process of Step S33 and later.

If it is determined in Step S32 that the mounting determination condition is not satisfied (N in Step S32), the process proceeds to Step S33 in which the count number of “No” is incremented by one. Then in Step S34, it is determined whether or not the count number of “No” is a predetermined value (e.g. 3) or more. If it is false (N in Step S34), the process proceeds to Step S35 in which the error flag is maintained. After Step S35, the process returns to Step S31.

In Step S34, if the count number of “No” is the predetermined value (Y in Step S34), the process proceeds to Step S36 in which the unmounted state is determined so that the error flag is set to one. Then, the process proceeds to Step S37 in which the count number of “No” is reset to zero, and the process returns to Step S31.

In addition, if it is determined in Step S32 that the mounting determination condition is satisfied (Y in Step S32), the process proceeds to Step S38 in which the mounted state is determined so that the error flag is set to zero. Then, after the count number of “No” is reset to zero in Step S37, the process returns to Step S31.

In the process of the second modified example illustrated in FIG. 22, if it is determined in Step S32 that the mounting determination condition is satisfied for a certain reason on the way of increasing the count number of “No” despite of the unmounted state actually, the mounted state is determined in Step S38, and the count number of “No” is reset to zero in Step S37. Therefore the condition for determining the unmounted state is more severe than that in the first modified example.

<Signal Measurement Example in Individual Ambient Environments>

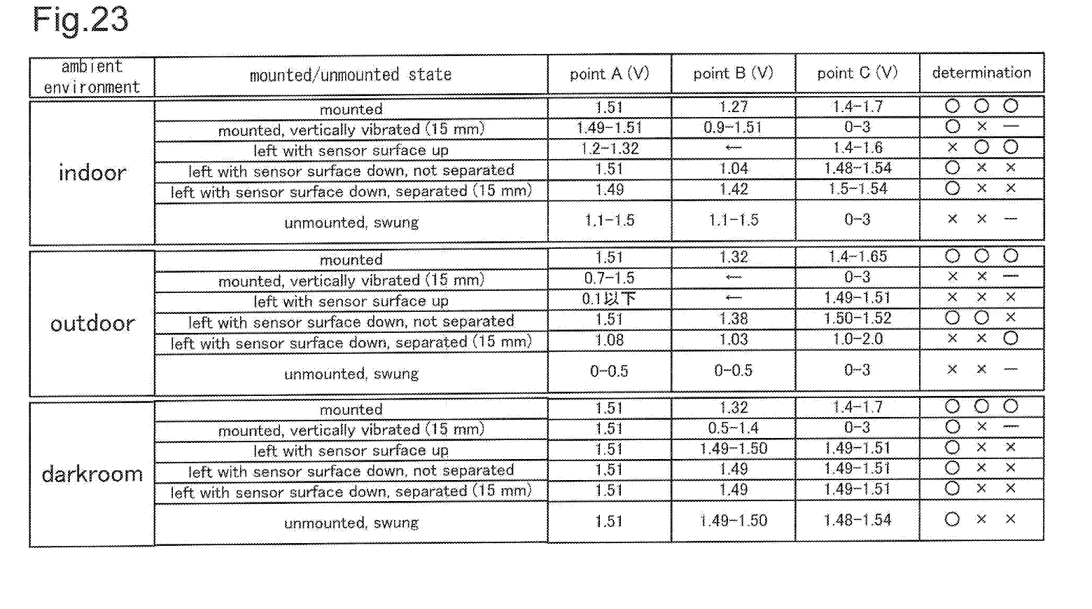

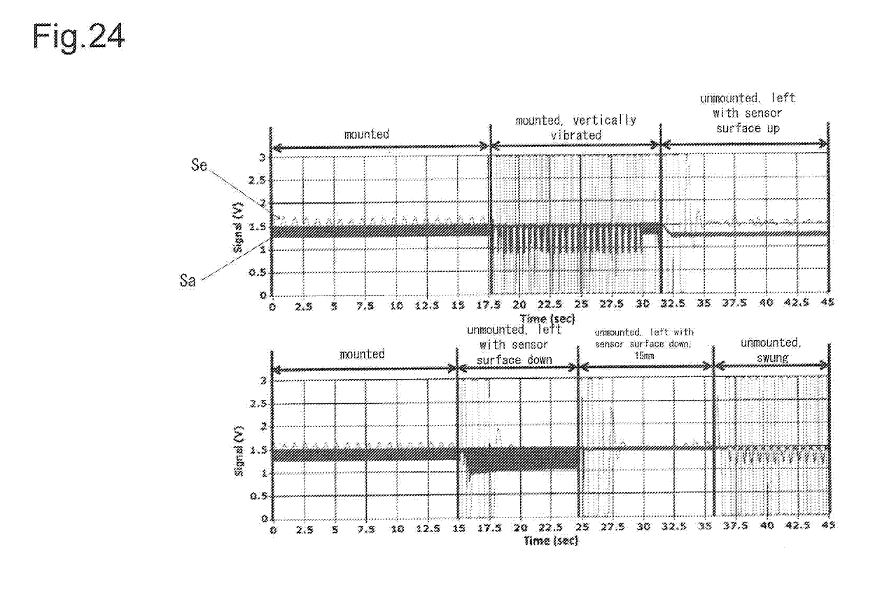

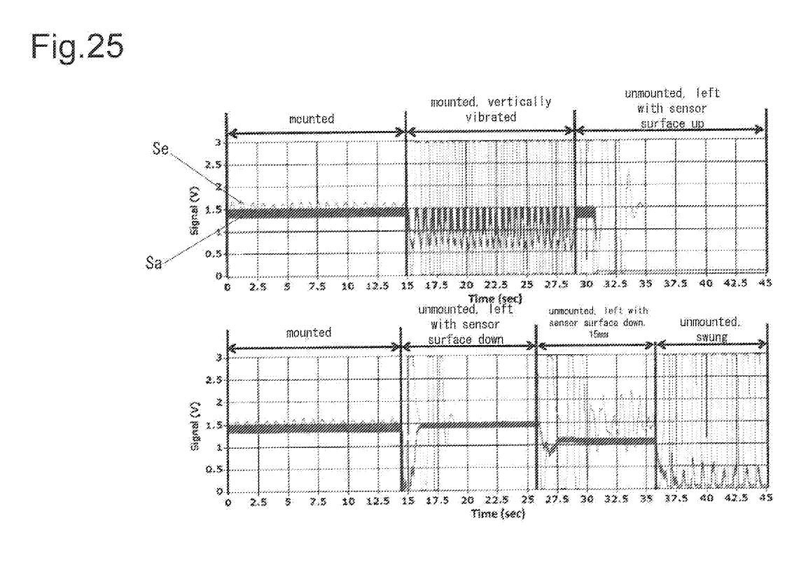

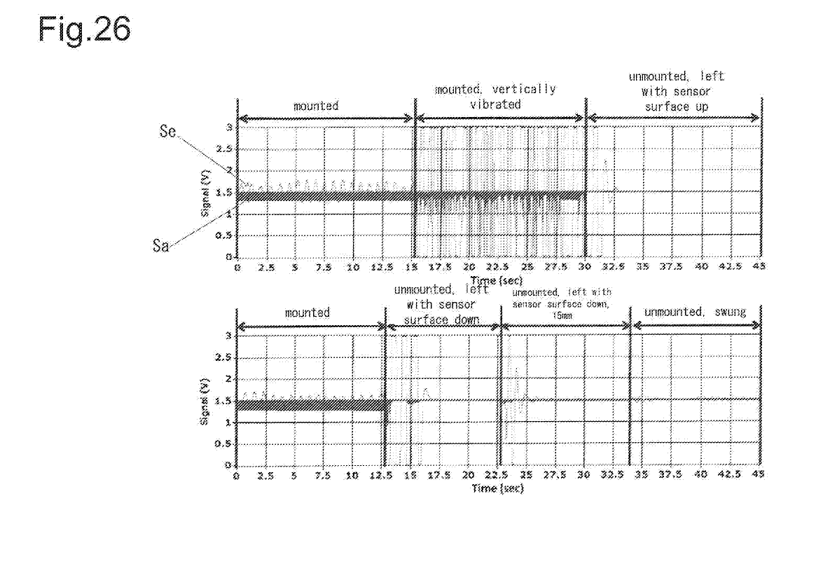

Here, in order to verify effectiveness of the determination of mounted/unmounted state, an example in which the signal was measured in individual ambient environments such as indoor, outdoor, and darkroom is shown in FIG. 23 as a list. In addition, a signal waveform example in individual mounting states measured indoors corresponding to FIG. 23 is illustrated in FIG. 24. In the same manner, waveform examples of the signal outdoors and in a darkroom are illustrated in FIGS. 25 and 26, respectively.

In FIG. 23, the column of “mounted/unmounted state” includes, from the upper row, a correctly mounted state, a mounted but coming off state, a state where the light sensor unit 11 is left on a desk with the light receiving surface up, a state where the light sensor unit 11 is left on a desk with the light receiving surface down, a state where the light sensor unit 11 is left on a desk with the light receiving surface down being separated from the desk surface, and a state where the pulse wave sensor 1 is held by hand and swung.

In addition, in FIG. 23, the “point A” indicates measured voltage value of the OFF voltage signal Sa@A, the “point B” indicates measured voltage value of the ON voltage signal Sa@B, and the “point C” indicates measured voltage value of the output signal Se. Note that if the voltage value varies, its variation range is shown, and “←” indicates to be the same value as the OFF voltage signal Sa@A.

Further in FIG. 23, the column of “determination” indicates the mounting determination results about, in order from the left, the OFF voltage signal Sa@A, the ON voltage signal Sa@B, and the output signal Se. The symbol “o” indicates determination of the mounted state, the symbol “x” indicates determination of the unmounted state, and the symbol “-” indicates the state where the signal is saturated (the state of fluctuating between the ground voltage and the power supply voltage). Note that the mounting determination condition is whether or not the OFF voltage signal Sa@A is the first threshold voltage of 1.4 V or higher, whether or not the ON voltage signal Sa@B is the third threshold voltage of 1.2 V or higher to the second threshold voltage of 1.4 V or lower, and whether or not there is timing when the output signal Se is the fourth threshold voltage of 1.6 V or higher.

As shown in FIG. 23, it is understood that the mounted state is determined for all signals in the correctly mounted state in each ambient environment of indoor, outdoor and darkroom, and in other states (in the unmounted state and in the abnormally mounted state) the unmounted state is determined for at least one of the signals, and hence detection of the mounted/unmounted state can be appropriately performed.

In particular, if the ambient environment is the darkroom, the mounted state is determined in all the unmounted state and the abnormally mounted state if only the OFF voltage signal Sa@A is used. Therefore the ON voltage signal Sa@B is also used for the determination, and hence correct determination can be made. Therefore, for example, for the purpose of supporting the darkroom, the determination may be made without using the output signal Se (Note that it is understood from FIG. 23 that the determination can be made by this method also indoors). However, as illustrated in FIG. 23, if the ambient environment is the outdoor, in the state where the light sensor unit 1 is left on the desk to face down, the mounted state is determined for both the OFF voltage signal Sa@A and the ON voltage signal Sa@B, and hence detection of the unmounted state can be correctly performed by adding the determination based on the output signal Se.

<Discussion About Output Wavelength>

In the experiment, using the so-called reflection type pulse wave sensor, there were examined behaviors when the output intensity (drive current value) of the light emitting unit was changed to 1 mA, 5 mA, and 10 mA while the output wavelength of the light emitting unit were λ1 (infrared: 940 nm), λ2 (green: 630 nm), and λ3 (blue: 468 nm). It is understood, as a result, in the visible light range of wavelengths at approximately 600 nm or less, an absorption coefficient of oxyhemoglobin HbO₂ is increased so that a peak intensity of the pulse wave to be measured is increased, and hence the waveform of the pulse wave can be obtained relatively easily.

Note that, in a pulse oximeter for detecting oxygen saturation in arterial blood, a wavelength in the near infrared region (approximately 700 nm), at which a difference between the absorption coefficient of the oxyhemoglobin HbO₂ (a solid line) and the absorption coefficient of the deoxyhemoglobin Hb (a broken line) becomes maximum, is widely and generally used as the output wavelength of the light emitting unit, but it can be said that, when considering use as a pulse wave sensor (in particular, as a so-called reflection type pulse wave sensor), as shown in the result of the experiment described above, it is desired to use the visible light range of wavelengths at 600 nm or less as the output wavelength of the light emitting unit.

However, when using a single light sensor unit for detecting both the pulse wave and the oxygen saturation in blood, in the same way as previous cases, it is possible to use a wavelength in the near infrared region.

<Other Variations>

Note that the various structures of the invention disclosed in this specification can be variously modified within the scope without deviating from the spirit of the invention, other than the embodiment described above. In other words, the embodiment is merely an example in every aspect and should not be interpreted as a limitation. The technical scope of the present invention is defined not by the above description of the embodiment but by the claims, and it should be understood to include all modifications within the equivalent meanings and scope of the claims.

### INDUSTRIAL APPLICABILITY

The various aspects of the invention disclosed in this specification can be used as techniques to improve convenience of a pulse wave sensor and a sleep sensor, and it is considered that the invention can be applied to various fields including health care support equipment, game equipment, music equipment, pet communication tools, doze prevention devices for vehicle drivers, and the like.

### EXPLANATION OF NUMERALS

1 pulse wave sensor

2 living body (wrist, ear, etc.)

10 main body unit

11 light sensor unit

11A light emitting diode

11B phototransistor

12 filter unit

121 transimpedance amplifier (current/voltage conversion circuit)

122 buffer circuit

123 detector circuit

124 band-pass filter circuit

125 amplifier circuit

126 reference voltage generation circuit

13 control unit

131 main control circuit

132 sub-control circuit

132a A/D converter

132b digital signal processing unit

132c serial data communication port (I²C port)

132d general input/output port (GPIO port)

14 display unit

15 communication unit

16 power supply unit

17 pulse drive unit

171 switch

172 current source

20 belt

AMP1 operational amplifier

R1 resistor

C1 capacitor

M1 pulse wave measurement module

1. A pulse wave sensor comprising:
a light sensor unit arranged to irradiate a living body with light from a light emitting unit and to detect reflected light or transmitted light from the living body by a light receiving unit, so as to generate a current signal corresponding to received light intensity;
a pulse drive unit arranged to turn on and off the light emitting unit at a predetermined frame frequency and duty;
a transimpedance amplifier arranged to convert the current signal into a voltage signal; and
a mounting determination unit arranged to perform mounting determination by comparing an OFF voltage signal obtained by the transimpedance amplifier during an OFF period of the light emitting unit with a predetermined first threshold voltage.

2. The pulse wave sensor according to claim 1, wherein the first threshold voltage is set to a voltage value lower than a reference voltage of the transimpedance amplifier.

3. The pulse wave sensor according to claim 1, wherein, the mounting determination unit performs the mounting determination by comparing an ON voltage signal obtained by the transimpedance amplifier during an ON period of the light emitting unit with a predetermined second threshold voltage and a predetermined third threshold voltage lower than the second threshold voltage.

4. The pulse wave sensor according to claim 3, further comprising a luminance adjustment control unit arranged to adjust luminance of the light emitting unit by comparing a voltage value based on the ON voltage signal obtained by controlling the pulse drive unit to turn on and off the light emitting unit with a predetermined threshold voltage for adjustment, wherein
the second threshold voltage is higher than the threshold voltage for adjustment, while the third threshold voltage is lower than the threshold voltage for adjustment.

5. The pulse wave sensor according to claim 3, wherein the mounting determination unit changes a first count number or a second count number according to whether or not both the OFF voltage signal and the ON voltage signal satisfy a mounting determination condition, so as to perform determination of mounted/unmounted state according to whether or not one of the first count number and the second count number has reached a predetermined value.

6. The pulse wave sensor according to claim 3, wherein the mounting determination unit changes a count number if at least one of the OFF voltage signal and the ON voltage signal does not satisfy a mounting determination condition, and otherwise resets the count number while performing determination of mounted state, and when the count number reaches a predetermined value, the mounting determination unit performs determination of unmounted state.

7. The pulse wave sensor according to claim 1, further comprising a signal output unit arranged to perform a process of extracting an envelope based on an output signal of the transimpedance amplifier so as to output a pulse wave signal, wherein
the mounting determination unit compares the pulse wave signal with a predetermined fourth threshold voltage so as to perform the mounting determination.

8. The pulse wave sensor according to claim 1, wherein the mounting determination unit monitors the OFF voltage signal a plurality of times at a predetermined sampling rate.

9. The pulse wave sensor according to claim 8, wherein the sampling rate is 1 to 8 Hz.

10. The pulse wave sensor according to claim 8, wherein the mounting determination unit compares each of the OFF voltage signals monitored a plurality of times during a predetermined determination period with the first threshold voltage, so as to perform the mounting determination based on all comparison results.

11. The pulse wave sensor according to claim 10, wherein the determination period is 1 to 5 seconds.

12. The pulse wave sensor according to claim 1, wherein the frame frequency is 50 to 1000 Hz.

13. The pulse wave sensor according to claim 1, wherein the duty is 1/8 to 1/200.

14. The pulse wave sensor according to claim 1, wherein the mounting determination unit outputs a result of the mounting determination via a general input/output port or a serial communication port.

15. The pulse wave sensor according to claim 1, wherein output wavelength of the light emitting unit is within a visible light range of 600 nm or less.

16. A pulse wave measurement module comprising:
a light sensor unit arranged to irradiate a living body with light from a light emitting unit and to detect reflected light or transmitted light from the living body by a light receiving unit, so as to generate a current signal corresponding to received light intensity;
a pulse drive unit arranged to turn on and off the light emitting unit at a predetermined frame frequency and duty;
a transimpedance amplifier arranged to convert the current signal into a voltage signal;
a signal output unit arranged to perform a process of extracting an envelope based on an output signal of the transimpedance amplifier so as to output a pulse wave signal;
a generation unit arranged to generate pulse wave information based on the pulse wave signal output from the signal output unit;
a mounting determination unit arranged to perform mounting determination by comparing an OFF voltage signal obtained by the transimpedance amplifier during an OFF period of the light emitting unit with a predetermined threshold voltage;
a first transmission unit arranged to externally transmit the pulse wave information generated by the generation unit; and
a second transmission unit arranged to externally transmit a result of the determination by the mounting determination unit.

17. The pulse wave measurement module according to claim 16, wherein the first transmission unit is a serial data communication port, while the second transmission unit is a serial data communication port or a general input/output port.
